(19) United States
(12) Patent Application Publication
(10) Pub. No.: US 2007/0173680 A1
(43) Pub. Date: Jul. 26, 2007

(54) Apparatus and method for performing therapeutic tissue ablation and brachytherapy

(75) Inventors: Robert F. Rioux, Ashland, MA (US); Paul DiCarlo, Middleboro, MA (US)

Correspondence Address: Vista IP Law Group LLP, 2040 MAIN STREET, 9TH FLOOR, IRVINE, CA, 92614, US

(73) Assignee: BOSTON SCIENTIFIC SCIMED, INC

(21) Appl. No.: 11/323,647

(22) Filed: Dec. 29, 2005

(51) Int. Cl. A61N 5/02; A61B 18/04; A61N 5/10

(52) U.S. Cl. 600/2; 378/65; 606/27

(57) ABSTRACT

A method, therapeutic probe, and system for treating a tissue margin surrounding an interstitial cavity is provided. The interstitial cavity may be created by resecting tissue, e.g., malignant tissue, from the patient's body to create the interstitial cavity. The interstitial cavity may assume any shape, but in a typical method, the interstitial cavity is spherically shaped. An expandable hyperthermic body of the therapeutic probe is expanded within the interstitial cavity into contact with the tissue margin. The tissue margin is heated with the hyperthermic body, and therapeutic x-ray radiation is conveyed from the hyperthermic body into the tissue margin. Heating of the tissue margin with the hypothermic body may ablate the tissue margin.

### FIELD OF THE INVENTION

The inventions relate generally to systems and methods for treating tissue, and in particular, the treatment of proliferative tissue, such as malignant tumors, using radiotherapy and hyperthermic therapy.

### BACKGROUND

It is known to treat proliferative tissue, such as cancerous tumors, using a surgical resection procedure. In a typical resection procedure, as much of the malignant tissue as possible is surgically cut from the patient's body, thereby creating an interstitial cavity. To prevent the infiltration of tumor cells, thereby limiting the therapeutic effect of the resection, it is typically common practice to supplement the resection procedure by targeting the tissue margin surrounding the interstitial cavity with radiation, with the goal of reducing its size or stabilizing it. Radiation therapy can be administered using one or more of a variety of techniques, including external-beam radiation, stereotactic radiosurgery, and brachytherapy. It is the latter that is pertinent to the claimed invention.

Brachytherapy can be performed by placing radiation sources (e.g., radioactive seeds) directly into the tissue to be treated, and when used in conjunction with surgical resection, within the interstitial cavity. To achieve the minimum prescribed dosage of radioactivity within the targeted tissue region, high activity radiation seeds are often used, resulting in the necrosis of healthy tissue, along with the malignant tissue. In order to facilitate a more uniform radiation exposure, thereby allowing the dosage of the radioactive seeds to be more tailored to the size of the interstitial cavity, it is known to expand a balloon around the radioactive source, so that the tissue margin is radially spaced about the radioactive source in a uniform manner. U.S. Pat. No. 6,413,204, which is fully and expressly incorporated herein by reference, describes such an apparatus.

Although the use of a balloon to facilitate the uniform application of radiation into the tissue margin surrounding an interstitial cavity is generally beneficial, certain regions of the tissue margin may not always conform to the balloon, thereby creating spaces or air gaps between the tissue margin and balloon, and resulting in a somewhat non-uniform application of radiation into the tissue margin.

Recently, it has been discovered that the use of hyperthermia (HT) therapy can be used as an adjunct to standard radiation therapy, such as brachytherapy, to increase the efficacy of the treatment. Hyperthermia can be defined as the treatment of disease by raising body temperature. When treating cancer, hyperthermia involves the use of heating devices (e.g., microwave applicators, ultrasound, low energy radio frequency conduction probes, or a sophisticated thermometry system of micro-thermocouples placed externally) in the natural cavities of the body, or in the case of surgical resection, interstially, to make cancerous tumors more operable, radiosensitive, or susceptible to cancer therapy measures. Hyperthermia can be applied prior to, during, and/or subsequent to the radiation therapy.

According to a study published in the May 1, 2005 edition of the Journal of Clinical Oncology, patients with post-mastectomy chest wall recurrence of breast cancer who were given HT therapy experienced complete response (total disappearance of the tumor) at a rate nearly three times higher than those patients who received radiation treatment alone. The use of adjuvant HT therapy also demonstrated a significant improvement in tumor control, among patients with recurrent melanoma as well as head and neck and other tumors when compared to stand-alone radiation therapy. It is thought that when combined with radiation therapy, HT therapy creates a mechanism that interferes with the cellular repair of radiation-induced DNA damage.

While it has been proven that the application of HT therapy facilitates the efficacy of standard radiation therapy, such as brachytherapy, separate tissue heating devices are needed, thereby complicating and generally increasing procedure time.

There thus remains a need to provide an integrated apparatus and method capable of applying brachytherapy/HT therapy to the tissue margin surrounding an interstitial cavity, while ensuring that the tissue margin uniformly surrounds the radiation source used during brachytherapy, thereby ensuring the uniform application of radiation to the tissue margin.

### SUMMARY OF THE INVENTION

In accordance with a first aspect of the present inventions, a method of treating a tissue margin surrounding an interstitial cavity is provided. The interstitial cavity may be created by resecting tissue, e.g., malignant tissue, from the patient's body to create the interstitial cavity. The interstitial cavity may assume any shape, but in a typical method, the interstitial cavity is spherically shaped.

The method comprises introducing a probe having an expandable hyperthermic body within the interstitial cavity, expanding the hyperthermic body within the interstitial cavity into contact with the tissue margin, heating the tissue margin with the hyperthermic body, and conveying therapeutic x-ray radiation from the hyperthermic body into the tissue margin. The x-ray radiation may originate from a location anywhere in or on the hyperthermic body, but in one method, the x-ray radiation originates from a location within an interior region of the hyperthermic body, e.g., at a location radially centered within the hyperthermic body. The tissue margin may be heated with the hyperthermic body prior to, or while, applying the therapeutic radiation from the hyperthermic body into the tissue margin. In any event, the same device that is used to apply x-ray radiation to the tissue margin is also conveniently used to heat the tissue margin, thereby increasing the therapeutic effect of the radiation therapy.

In an optional method, heating of the tissue margin with the hypothermic body ablates the tissue margin. By way of non-limiting example, ablation of the tissue margin conforms the interstitial cavity to the shape of the expanded hypothermic body, which removes air gaps between the hyperthermic body and the tissue margin, thereby providing for a more uniform application of the therapeutic radiation. In this case, ablation of the tissue margin is performed prior to the application of therapeutic radiation. The tissue margin may also be ablated by the hyperthermic body subsequent to applying the therapeutic radiation, e.g., to necrose any remaining malignant tissue.

In accordance with a second aspect of the present inventions, a therapeutic probe for treating tissue within a patient's body is provided. The therapeutic probe comprises an elongate shaft, a radioactive source configured for being located at a distal end of the shaft, and an expandable hyperthermic body carried by a distal end of the elongate shaft and surrounding the distally located radioactive source. The hyperthermic body is configured for radially conveying heat and radiation from the radioactive source into the tissue.

In one embodiment, the hyperthermic body is configured to conform with an interstitial cavity. For example, the hyperthermic body may be self-expandable. In this case, the hyperthermic body may comprises a foam electrode body, and the elongate shaft may comprise at least one fluid lumen in communication with the foam electrode body. The hyperthermic body, when expanded, may assume any suitable shape, but in one embodiment, is spherically-shaped. In an optional embodiment, the hyperthermic body is configured for radially conveying ablation energy into the tissue. Ablation of the tissue may be advantageous for the same reasons discussed above.

The radioactive source may be configured for being located anywhere in or on the hyperthermic body, but in one embodiment, is configured for being located within a radial center of the hyperthermic body. In another embodiment, the therapeutic probe comprises an elongated element carrying the radioactive source, and the elongated shaft comprises a delivery lumen configured for receiving the elongated element. The therapeutic probe may comprise an electrical connector carried by a proximal end of the elongated shaft, wherein the hyperthermic body comprises an electrode electrically coupled to the electrical connector.

In accordance with a third aspect of the present inventions, a system for treating tissue within a patient's body is provided. The system comprises the therapeutic probe described above, and a source of thermal energy coupled to the hyperthermic body.

Other and further aspects and features of the invention will be evident from reading the following detailed description of the preferred embodiments, which are intended to illustrate, not limit, the invention.

### BRIEF DESCRIPTION OF DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a plan view a tissue treatment system constructed in accordance with a preferred embodiment of the present invention;

FIG. 2 is a partially cutaway side view of a tissue treatment probe used in the tissue ablation system of FIG. 1, wherein a tissue ablation body is shown in a collapsed low-profile geometry;

FIG. 3 is a partially cutaway side view of the tissue treatment probe of FIG. 2, wherein the tissue ablation body is shown in an expanded geometry;

FIG. 4 is a cross-section view of the tissue treatment probe of FIG. 3, taken along the line 4-4; and

FIGS. 5A-5G are side views illustrating a method of treating tissue using the tissue ablation system of FIG. 1.

### DETAILED DESCRIPTION OF THE ILLUSTRATED EMBODIMENTS

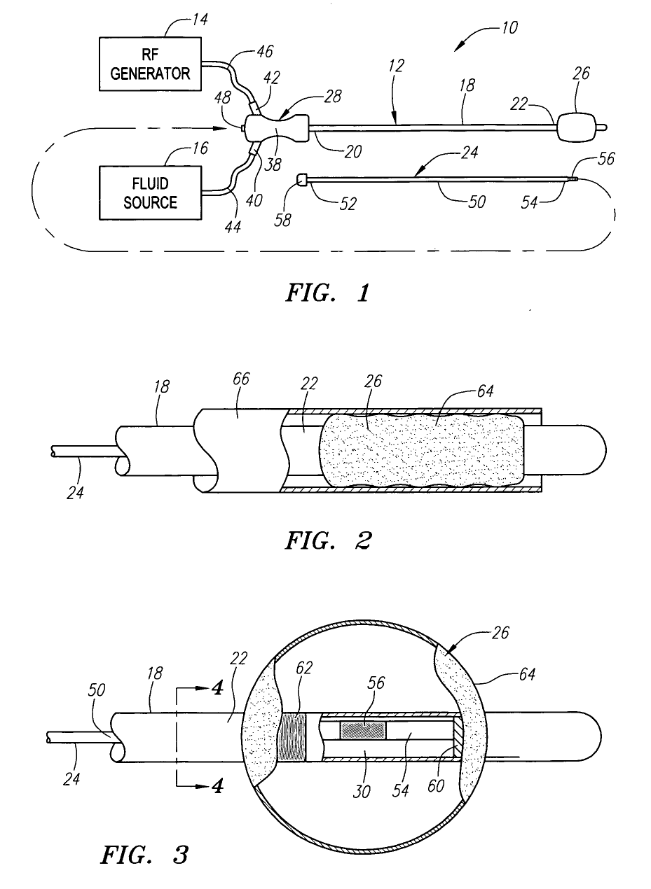

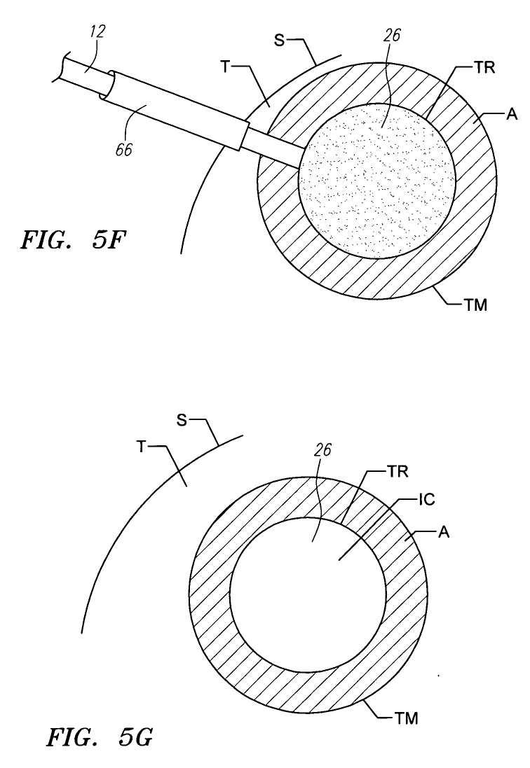

Referring generally to FIG. 1, a tissue treatment system 10 constructed in accordance with one embodiment of the present inventions will be described. The tissue treatment system 10 generally includes a treatment probe 12, an ablation source 14, and in particular, a radio frequency (RF) generator, and a source of electrically conductive fluid 16. The treatment probe 12 can be introduced into the body of a patient to necrose proliferative tissue using X-ray radiation in conjunction with hyperthermia therapy (HT), and in particular, RF ablation therapy. In the illustrated embodiment, the treatment probe 12 is configured to be introduced into and operated to treat the tissue margin surrounding an interstitial cavity (shown in FIG. 5) from which a malignant tumor has been resected.

As will be further appreciated below, ablation energy can be conveyed from the treatment probe 12 in order to pre-condition the tissue margin, and in particular, to elevate the temperature within the tissue margin, as well as to physically mold or fix the interstitial cavity into a desired and uniform shape. The same treatment probe 12 can then be operated to emit X-ray radiation into the tissue margin in a uniform manner, thereby necrosing most, if not all, of the malignant tissue within the tissue margin. The elevated temperature of the tissue margin resulting from the previous ablation step increases the therapeutic effect of the X-ray radiation treatment. During the X-ray radiation delivery, the treatment probe 12 can also be operated to maintain the elevated temperature of the tissue margin, thereby ensuring that the combined radiation/HT therapy is continued. Lastly, the treatment probe 12 can be operated to further ablate the tissue margin, thereby ensuring that all of the malignant tissue within the tissue margin is necrosed, as well as to further continue the combined effect of the radiation/HT therapy.

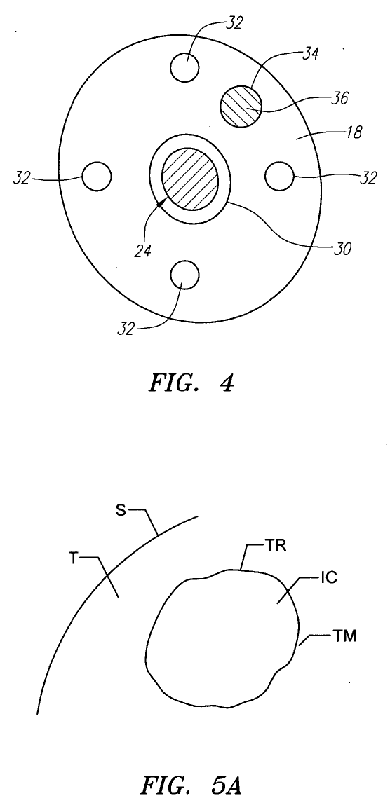

Referring further to FIGS. 2-4, the detailed features of the probe 12 used to perform the aforementioned functions will now be discussed. The probe 12 generally includes an elongate shaft 18 having a proximal end 20 and a distal end 22, a deliverable radioactive mechanism 24 and an expandable hyperthermic body 26, and specifically a tissue ablation body, carried by the distal shaft end 22, and a handle assembly 28 carried by the proximal shaft end 20.

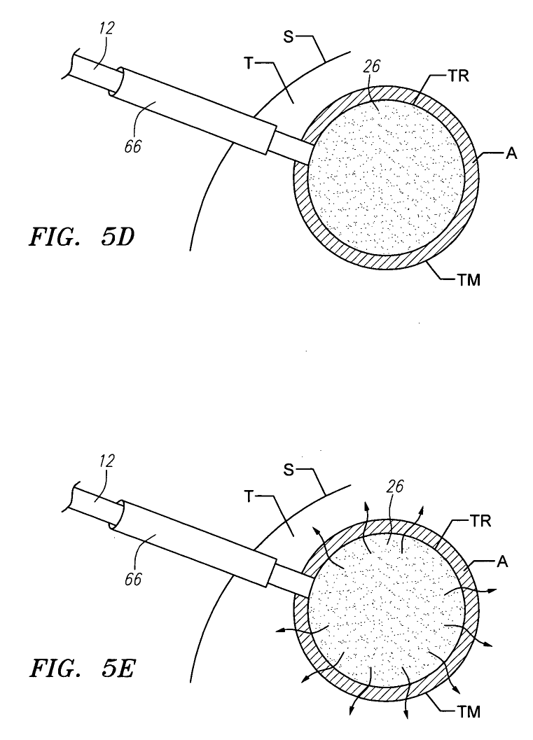

The probe shaft 18 may be rigid, semi-rigid, or flexible depending upon the designed means for introducing the probe 12 into the interstitial cavity, and may be composed of any suitable biocompatible material. As best shown in FIG. 4, the probe shaft 18 includes a central delivery lumen 30 for slidably receiving the radioactive mechanism 24, a plurality of fluid delivery lumens 32 for conveying electrically conductive fluid to the expandable ablation body 26, and an ablation wire lumen 34 for carrying a radio frequency (RF) wire 36 that will be in electrical communication with the expandable ablation body 26. The lumens can be formed within the probe shaft 18 using standard means, such as extrusion.

The handle assembly 28 includes a handle sleeve 38 shaped in a manner that facilitates grasping by a physician or medical technician. The handle sleeve 38 is preferably composed of a durable and rigid material, such as medical grade plastic, and is ergonomically molded to allow a physician to more easily manipulate the treatment probe 12. The handle assembly 28 further includes a fluid infusion port 40 in fluid communication with the ablation body 26 via the fluid delivery lumens 32 carried by the probe shaft 18, and an electrical connector 42 electrically coupled to the ablation body 26 via the RF wire 36 carried by the probe shaft 18. The infusion port 40 is configured for mating with the proximal end of a fluid conduit 44 connected to the fluid source 16. The electrical connector 42 is configured for mating with the proximal end of a RF cable 46 connected to the RF generator 14. Alternatively, the RF cable 46 may be hardwired within the handle sleeve 38. Lastly, the handle assembly 28 includes a locking member 48 configured for mating with the radioactive mechanism 24, as will be described in further detail below.

The radioactive mechanism 24 includes an elongated shaft 50 having a proximal end 52 and a distal end 54, a radioactive source 56 carried by the distal end 54 of the elongated shaft 50, and a locking member 58 carried by the proximal end 52 of the elongated shaft 50. The elongated shaft 50 may be composed of any rigid or semi-rigid material, such as stainless steel, that provides the shaft 50 with the column strength sufficient to introduce the radioactive source 56 through the delivery lumen 30 of the probe shaft 18. The radioactive source 56 can be composed of any suitable solid X-ray radiation emitting material, such as Palladium-103 or Iodine-125. To ensure that only the tissue surrounding the ablation body 26 is exposed to X-ray radiation, a radiation shielding (not shown) can be applied to the exterior surface of the probe shaft 18, e.g., by coating, proximal to the ablation body 26.

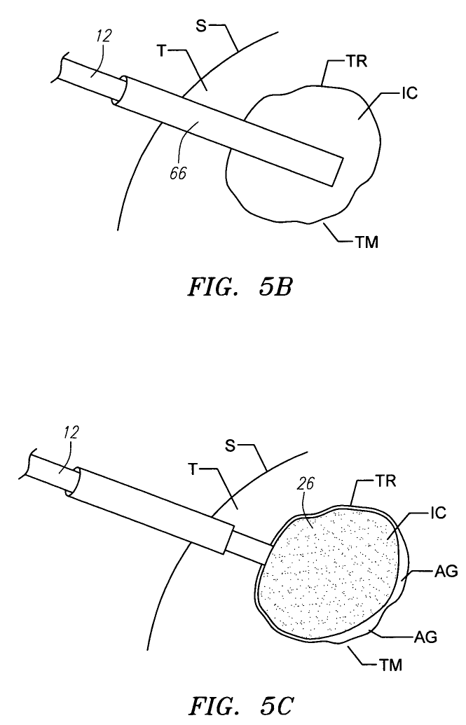

The shaft 50 of the radioactive mechanism 24 is dimensioned and configured, such that radioactive source 56 is situated in the radial center and axial center of the ablation body 26 when the radioactive mechanism 24 is completely located within the delivery lumen 30 of the probe shaft 18. To ensure that the radioactive mechanism 24 does not extend past the desired axial location, the probe shaft 18 includes a stopper 60 (shown best in FIG. 3) located at the distal end of the delivery lumen 30, so that the distal shaft end of the radioactive mechanism 24 abuts the stopper 60. The locking members 48 and 58 of the handle assembly 28 and radioactive mechanism 24 are configured to engage each other once the radioactive mechanism 24 is completely disposed within the delivery lumen 30. For example, the locking members 48, 58 may comprise complementary thread arrangements.

The expandable ablation body 26 surrounds the radioactive source 56 when the radioactive mechanism 24 is completely located within the delivery lumen 30 of the probe shaft 18, and is constructed in a manner that allows it to both expand and convey ablation energy, and in particular, RF energy. In the illustrated embodiment, the ablation body 26 comprises an internal electrode 62 (shown best in FIG. 3) that is disposed about the distal end 22 of the probe shaft 18, and an outer expandable/compressible electrode body 64. The shape of the ablation body 26 will ultimately depend on the shape of the interstitial cavity that is to be treated. In the illustrated embodiment, the ablation body 26 is spherical, which is the typical shape that an interstitial cavity will have.

The internal electrode 62 can be composed of any electrically conductive material, such as gold, platinum, etc., and can be formed onto the probe shaft 18 using any suitable manner, such as coating, sputtering, etc., or by forming the internal electrode 62 as a discrete element that can then be mounted to the probe shaft 18 using, e.g., an interference fit. The internal electrode 62 is suitably coupled to the RF wire 36 extending through the RF wire lumen 34 of the probe shaft 18, so that the electrical connector 42 located on the handle assembly 28 is in electrical communication with the internal electrode 62. To provide exterior access to the RF wire 36 from the internal electrode 62, the distal end of the RF wire 36 can extend through an access port (not shown) transversely formed through the wall of the probe shaft 18.

The outer electrode body 64 can be composed of any material that allows it to radially convey ablation energy from the internal electrode 62 outward into any surrounding tissue, while also allowing the passage of radiation from the radioactive source 56 into the surrounding tissue. In the illustrated embodiment, the outer electrode body 64 is composed of a foam material. Suitable materials that can be used to construct the outer electrode body 64 include open-cell foam (such as polyethylene foam, polyurethane foam, polyvinylchloride foam) and medical-grade sponges. Further details regarding exemplary constructions of foam electrode bodies are disclosed in U.S. patent application Ser. No. 11/xxx,xxx (Attorney Docket No. 28-7045232001), entitled “Compressible/Expandable Hydrophilic Ablation Electrode”, which is fully and expressly incorporated herein by reference.

It can be appreciated that the foam outer electrode body 64 is self-expandable. That is, the outer electrode body 64 will expand in the absence of a compressive force. An outer sheath 66 (shown in FIG. 2) can be used to selectively place the outer electrode body 64 into a low-profile geometry, e.g., by distally sliding the outer sheath 66 to apply a compressive force to the electrode body 64, and allow the outer electrode body 64 to expand into an expanded geometry, e.g., by proximally sliding the outer sheath 66 to release the compressive force from the outer electrode body 64.

The outer electrode body 64 is in fluid communication with the fluid delivery lumens 32 extending the probe shaft 18, so that the infusion port 40 located on the handle assembly 28 is in fluid communication with outer electrode body 64. To provide exterior access to fluid delivery lumens 32, access ports (not shown) are transversely formed through the wall of the probe shaft 18. Thus, it can be appreciated that when the outer electrode body 64 is saturated with an electrically conductive fluid, such as saline, an electrical path is formed through the outer electrode body 64, so that electrical energy in the form of RF energy can be transmitted from the internal electrode, through the outer electrode body 64, into any tissue surrounding the ablation body 26.

It should be appreciated that the ablation body 26 may have constructions other than that illustrated in FIGS. 1-4 as long as the ablation body 26 is capable of both delivering ablation energy and X-ray radiation. For example, instead of using a solid radioactive source 56 located at the distal end of wire, the radiation source can take the form of a liquid contained within a chamber within the center of the outer electrode body 64. Such an arrangement is disclosed in U.S. Pat. No. 6,413,204, which has previously been incorporated herein by reference. As another example, instead of using an outer electrode body composed of a foam material, the outer electrode body can include an inflatable microporous balloon such as those described in U.S. Pat. No. 5,722,403, which is expressly incorporated herein by reference. Inflation of the microporous balloon into an expanded geometry can be accomplished using the fluid source 16. In this case, the fluid conveyed through the fluid delivery lumens 32 within the probe shaft 18 can be used as an inflation medium to both inflate the balloon, while also providing a means for conducting ablation energy from the internal electrode 62, through pores within the wall of the balloon, into the surrounding tissue.

Referring back to FIG. 1, the RF generator 14 may be a conventional general purpose electrosurgical power supply operating at a frequency in the range from 300 kHz to 5 MHz, with a conventional sinusoidal or non-sinusoidal wave form. Such power supplies are available from many commercial suppliers, such as Valleylab, Aspen, Bovie, and Ellman. Most general purpose electrosurgical power supplies, however, are constant current, variable voltage devices and operate at higher voltages and powers than would normally be necessary or suitable. Thus, such power supplies will usually be operated initially at the lower ends of their voltage and power capabilities, with voltage then being increased as necessary to maintain current flow. More suitable power supplies will be capable of supplying an ablation current at a relatively low fixed voltage, typically below 200 V (peak-to-peak). Such low voltage operation permits use of a power supply that will significantly and passively reduce output in response to impedance changes in the target tissue. The output will usually be from 5 W to 300 W, usually having a sinusoidal wave form, but other wave forms would also be acceptable. Power supplies capable of operating within these ranges are available from commercial vendors, such as Boston Scientific Therapeutics Corporation. Preferred power supplies are model RF-2000 and RF-3000, available from Boston Scientific Corporation.

RF current is preferably delivered from the RF generator 14 to the ablation body 26 in a monopolar fashion, which means that current will pass from the ablation body 26, which is configured to concentrate the energy flux in order to have an injurious effect on the adjacent tissue, and a dispersive electrode (not shown), which is located remotely from the ablation body 26, and has a sufficiently large area (typically 130 cm² for an adult), so that the current density is low and non-injurious to surrounding tissue. In the illustrated embodiment, the dispersive electrode may be attached externally to the patient, e.g., using a contact pad placed on the patient's flank.

The electrically conductive fluid source 16 may take the form of any device capable of introducing an electrically conductive fluid into the infusion port 40 under a positive pressure. For example, the fluid source 16 may take the form of a syringe or an automated pump assembly.

Having described the structure of the tissue treatment system 10, its operation in treating target tissue will now be described. The target tissue may be located anywhere in the body where radiation/hyperthermic exposure may be beneficial. Most commonly, the treatment region will be located within an organ of the body, such as the liver, kidney, pancreas, breast, prostrate (not accessed via the urethra), and the like. The tissue treatment system 10 particularly lends itself well to the treatment of a tissue margin surrounding an interstitial cavity resulting from the removal of a volume of malignant tissue. The volume of the interstitial cavity will ultimately depend on the size of the tumor or other tissue resected from the patient's body, typically being in the range of 1 cm³ to 150 cm³, and often from 2 cm³ to 35 cm³.

The shape of the interstitial cavity will typically be spherical to match the spherical profile of the radiation energy used to treat the tissue margin, thereby minimizing the volume of healthy tissue that is necrosed. The treatment region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like. Preferred is the use of high resolution ultrasound of the tumor or other lesion being treated, either intraoperatively or externally.

Referring now to FIGS. 5A-5G, the operation of the tissue treatment system 10 is described in treating a treatment region TR beneath the skin or an organ surface S of a patient. Although a single treatment region TR is illustrated for purposes of brevity, the tissue treatment system 10 may alternatively be used to treat multiple treatment regions TR. The treatment region TR comprises an interstitial cavity IC and a tissue margin TM surrounding the interstitial cavity IC. The interstitial cavity IC can be created in a standard manner, e.g., removal of malignant tissue from the patient's body. As illustrated in FIG. 5A, the interstitial cavity IC, while generally spherical, has some spatial irregularities that could result in the presence of gaps or air pockets between the expanded ablation body 26 and the tissue margin TM, as discussed below.

After the treatment region TR has been resected to create the interstitial cavity IC, the probe 12 is introduced through the tissue T in a standard manner, so that the ablation body 26 (not shown) is located within the interstitial cavity IC (FIG. 5B). Because the ablation body 26 is self-expandable, the outer sheath 66 is used to compress the ablation body 26 into its low-profile geometry while the probe 12 is introduced through the tissue T. Alternatively, if an inflatable ablation body, such as a microporous balloon, is used, the probe 12 will be introduced through the tissue T while the ablation body 26 is deflated. Once the ablation probe 12 is properly positioned, the outer sheath 66 is displaced in the proximal direction, thereby releasing the compressive force and allowing the ablation body 26 to self-expand into its expanded geometry within the interstitial cavity IC (FIG. 5C). If an inflatable ablation body is used, a source of inflation medium, such as the fluid source 16, can be used to convey an inflation medium through the probe shaft into the hyperthermic body. As can be seen, although the expanded ablation body 26 and the interstitial cavity IC are generally spherical, the ablation body 26 does not conform exactly to the interstitial cavity IC to the extent that air gaps AG exist between the ablation body 26 and the tissue margin TM.

Next, the fluid source 16 is coupled to the infusion port 40 located on the handle assembly 28 (shown in FIG. 1), and operated, such that electrically conductive fluid is conveyed through the fluid delivery lumens 32 located along the probe shaft 18, and into the expandable/collapsible outer electrode body 64. To the extent that the outer electrode body 64 does not fully expand upon the release of the compressive force, the fluid will be absorbed by the outer electrode body 64, thereby ensuring that the ablation body 26 is fully expanded within the interstitial cavity IC. The RF generator 14 is then connected to the electrical connector 42 located on the handle assembly 28, and operated, such that RF energy is conveyed along the RF generator 14, along the RF wire 36, and to the expanded ablation body 26, thereby resulting in the ablation A of the surrounding tissue margin TM (FIG. 5D). As can be seen, ablation of the tissue margin TM smoothes outer the outer periphery of the interstitial cavity IC, thereby providing a nearly perfect spherical shape that conforms to the spherical shape of the expanded ablation body 26. In addition, the temperature of the tissue margin TM becomes elevated, thereby preconditioning the tissue margin TM for subsequent radiation treatment, as discussed below.

Next, the radiation mechanism 24 is inserted within the delivery lumen 30 of the treatment probe 12 until the distal end of the radiation mechanism 24 abuts the stopper 60 at the end of the delivery lumen 30 (shown in FIG. 3), thereby axially centering the radioactive source 56 within the ablation body 26. As a result, x-ray radiation (shown as arrows) is emitted from the radioactive source 56, through the ablation body 26, and into the tissue margin TM, thereby necrosing the deeper tissue not otherwise necrosed by the initial RF ablation (FIG. 5E). Notably, the elevated temperature of the tissue margin TM resulting from the initial RF ablation facilitates the therapeutic effect of the radiation therapy, as previously discussed. Optionally, the RF generator may be operated during the radiation therapy, such that the tissue margin TM is heated, thereby maintaining the elevated temperature of the tissue margin TM.

Next, the RF generator 14 is again operated to necrose any tissue in the tissue margin TM that has not been necrosed by the radiation therapy, thereby expanding the ablation region A (FIG. 5F). Alternatively, the RF generator 14 may be operated, such that the tissue margin TM is heated, but tissue ablation does not result from such heating. In either case, to the extent that the temperature of the tissue margin TM has decreased due to the lapse of time from the initial tissue ablation, the temperature of the tissue margin TM is again elevated to increase the therapeutic effect of the previously performed radiation therapy. Next, the treatment probe 12 is removed from the patient's body, leaving behind the interstitial cavity IC surrounded by an ablated and radiation treated tissue margin TM (FIG. 5G). Alternatively, the treatment probe 12 may be left within the patient's body, so that subsequent cycles of radiation/hyperthermic therapy can be performed without reintroducing the probes within the patient's body.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed:

1. A method of treating a tissue margin surrounding an interstitial cavity within a patient's body, comprising:
introducing a probe having an expandable hyperthermic body within the interstitial cavity;
expanding the hyperthermic body within the interstitial cavity into contact with the tissue margin;
heating the tissue margin with the hyperthermic body;
conveying therapeutic x-ray radiation from the hyperthermic body into the tissue margin.

2. The method of claim 1, further comprising resecting tissue from the patient's body to create the interstitial cavity.

3. The method of claim 2, wherein the resected tissue comprises malignant tissue.

4. The method of claim 1, wherein the interstitial cavity is spherically shaped.

5. The method of claim 1, wherein the tissue margin is heated with the hyperthermic body prior to applying the therapeutic radiation from the hyperthermic body into the tissue margin.

6. The method of claim 1, wherein the tissue margin is heated with the hyperthermic body while applying the therapeutic radiation from the hyperthermic body into the tissue margin.

7. The method of claim 1, wherein heating of the tissue margin with the hyperthermic body ablates the tissue margin.

8. The method of claim 7, wherein ablation of the tissue margin conforms the interstitial cavity to the shape of the expanded hyperthermic body.

9. The method of claim 7, wherein the tissue margin is ablated by the hyperthermic body subsequent to applying the therapeutic radiation from the hyperthermic body into the tissue region.

10. The method of claim 1, wherein heating of the tissue margin increases a therapeutic effect of the x-ray radiation treatment of the tissue margin.

11. The method of claim 1, wherein the x-ray radiation originates from a location within an interior region of the hyperthermic body.

12. The method of claim 1, wherein the x-ray radiation originates from a location radially centered within the hyperthermic body.

13. A therapeutic probe for treating tissue within a patient's body, comprising:
an elongate shaft;
a radioactive source configured for being located at a distal end of the shaft; and
an expandable hyperthermic body carried by a distal end of the elongate shaft and surrounding the distally located radioactive source, the hyperthermic body configured for radially conveying heat and radiation from the radioactive source into the tissue.

14. The therapeutic probe of claim 13, wherein the hyperthermic body comprises a foam electrode body, and the elongate shaft comprises at least one fluid lumen in communication with the foam electrode body.

15. The therapeutic probe of claim 13, wherein the hyperthermic body is self-expandable.

16. The therapeutic probe of claim 13, wherein the hyperthermic body, when expanded, is spherically-shaped.

17. The therapeutic probe of claim 13, wherein the hyperthermic body is configured for radially conveying ablation energy into the tissue.

18. The therapeutic probe of claim 13, wherein the radioactive source is configured for being located within a radial center of the hyperthermic body.

19. The therapeutic probe of claim 13, further comprising an elongated element carrying the radioactive source, wherein the elongated shaft comprises a delivery lumen configured for receiving the elongated element.

20. The therapeutic probe of claim 13, further comprising an electrical connector carried by a proximal end of the elongated shaft, wherein the hyperthermic body comprises an electrode electrically coupled to the electrical connector.

21. A system for treating tissue within a patient's body, comprising:
the therapeutic probe of claim 13; and
a source of thermal energy coupled to the hyperthermic body.
